(19) United States
(12) Patent Application Publication
(10) Pub. No.: US 2014/0058446 A1
(43) Pub. Date: Feb. 27, 2014

(54) SPINAL IMPLANT SYSTEM

(71) Applicant: Avi Bernstein, Chicago, IL (US)

(72) Inventor: Avi Bernstein, Chicago, IL (US)

(21) Appl. No.: 14/072,459

(22) Filed: Nov. 5, 2013

Related U.S. Application Data
(63) Continuation-in-part of application No. 13/247,522, filed on Sep. 28, 2011.

(51) Int. Cl. A61B 17/70; A61B 19/00

(52) U.S. Cl. CPC A61B 17/7059; A61B 19/46
USPC 606/246; 606/102; 29/428

(57) ABSTRACT

A spinal implant system and method of assembling the system. The system includes an implant, an integral holder/connector to grasp the implant and to connect to a plate before the assembly is inserted into a patient.

### PRIORITY CROSS-REFERENCE TO RELATED APPLICATION

This application is a continuation-in-part and claims priority pursuant to 35 U.S.C. 119(e) from U.S. patent application Ser. No. 13/247,522, filed on Sep. 28, 2011, which is expressly incorporated herein by reference.

### FIELD OF THE INVENTION

The present invention relates generally to a spinal implant system and, more particularly, to a spinal implant system that is modular and easily assembled before insertion into a patient.

### BACKGROUND OF THE INVENTION

Spinal implants and hardware are known and have been disclosed in several earlier patents. By way of example, in 2007, a U.S. Pat. No. 7,172,627, issued to Fiere, Fayda and Taziaux for a “Stabilized Interbody Fusion System for Vertebrae” purports to disclose an implant/cage 4, an assembly screw 22, a connected stabilizing plate 17, and screws 19 for attachment to vertebrae. In the same year a patent issued to Ross, U.S. Pat. No. 7,306,605, and purports to disclose a plate with locking elements. A year later, U.S. Patent Application Publication No., 2008/0161925 appeared listing Brittan and Smith as inventors and entitled “Articulating Spinal Implant.” The Application discloses a spacer 12 pivotally connected to a plate 14 with fasteners 34 used to connect the combination to vertebrae.

In 2009, Patent Application Publication No., 2009/0306779 appeared listing Ahn as inventor and entitled “Modular Anterior Locking Interbody Cage.” The Ahn Application discloses an implant 305, a plate 310, an attachment screw 335 between the implant and the plate, and vertebrae attachment screws 325. A patent to Kirschman, U.S. Pat. No. 7,641,701, issued in 2010 and entitled “Spinal Fusion System and Method for Fusing Spinal Bones,” purports to disclose a fusion system 24 including a cover 42, implants including a housing 32 and graft material 38, rails 48, 50 to receive the cover, and screws 46. An Application was published in 2010, No., 2010/0249937, listing Blain and others entitled “Flanged Interbody Fusion Device,” that appears similar to the disclosure in the Brittan and Smith Application and discloses a spacer 114 pivotally connected to a plate 102 with fasteners 158 used to connect the combination to vertebrae. An Application published in 2011, entitled “Combined Spinal Interbody and Plate Assemblies, No., 2011/0015745, listing Bucci as the inventor purports to disclose a spinal spacer 12 pivotally connected to a divided spine plate 14 with the combination being attached to vertebrae using bone screws 16.

The inventions discussed in connection with the described embodiments below address deficiencies of the prior art. The features and advantages of the present inventions will be explained in, or become apparent from, the following summary and description of the preferred embodiments considered together with the accompanying drawings.

### SUMMARY OF THE INVENTION

In accordance with the present invention, an advantageous method and system are described in the form of a spinal implant system and the steps of assembling the system. The system is flexible, efficient, simple and cost effective. The spinal implant system is simply constructed, structurally robust, compact, and yet relatively inexpensive. The present invention also includes a workstation and a depth indicator instrument.

Briefly summarized, the invention relates to a spinal implant system including a holder having two adjustable legs for engaging an implant, a plate for engagement with vertebrae, a connector connected at one end to the holder and extending a predetermined distance to connect at an opposite end to the plate, and a plurality of screws positioned through the plate for fastening the plate to vertebrae wherein the implant, the holder, the connector and the plate are assembled before insertion of the spinal implant system into a patient.

The invention also relates to a method of assembling the spinal implant system including the steps of forming a workstation with two spaced apart supports, placing an implant between the supports, mounting a holder to the implant, the holder including an integral connector, and mounting the plate to the connector. The method may also include the steps of tightening the holder onto the implant and adjusting the connector after measuring the depth of the implant.

### BRIEF DESCRIPTION OF THE DRAWINGS

For the purpose of facilitating an understanding of the invention, the accompanying drawings and detailed description illustrate embodiments thereof, from which the structures, the construction and operation, the processes, and many related advantages of the embodiments may be readily understood and appreciated.

FIG. 1 is an exploded isometric view of a spinal implant, a holder/connector, and a plate with screws.

FIG. 2 is an isometric view of the implant, the holder/connector and the plate shown in FIG. 1 attached to vertebrae of a spine.

FIG. 3 is a plan view of an integral holder/connector with a ratchet mechanism connected to a plate.

FIG. 4 is a partial elevation view of the integral holder/connector shown in FIG. 3.

FIG. 5 is a plan view of another integral holder/connector embodiment.

FIG. 6 is a partial plan view of a leg of yet another integral holder/connector embodiment similar to that shown in FIG. 5.

FIG. 7 is a diagrammatic elevation view illustrating an implant, a holder, a connector and a plate with screws wherein there is an articulation at the plate/connector junction.

FIG. 8 is a diagrammatic front elevation view of the embodiment shown in FIG. 7.

FIG. 9 is a diagrammatic elevation view of an implant, a holder, a connector and a plate wherein there is an articulation at the connector/holder junction.

FIG. 10 is a diagrammatic front elevation view of a plate and an adjustable holder/connector.

FIG. 11 is a diagrammatic side elevation view illustrating an implant, the adjustable holder/connector, and the plate of the embodiment shown in FIG. 10.

FIG. 12 is a diagrammatic plan view of an implant, a holder, a connector and a plate with articulations between a holder base and holder legs, articulations between the holder base and connector arms, and articulations between the connector arms and the plate.

FIG. 13 is a diagrammatic side elevation view of an implant between vertebrae but spaced from a spinal cord, a holder and connector, a plate and screws in adjacent vertebrae.

FIG. 14 is a diagrammatic plan view of an instrument and a spacer for measuring the depth of an implant.

FIG. 15 is a diagrammatic plan view of forceps.

FIG. 16 is a diagrammatic plan view of a mallet.

FIG. 17 is a diagrammatic isometric view of an instrument case.

FIG. 18 is a diagrammatic isometric view of a tray having instruments and a workstation.

FIG. 19 is an enlarged section view taken along line 19-19 of FIG. 18.

FIG. 20 is a diagrammatic isometric view of a smaller tray with additional instruments.

FIG. 21 is a diagrammatic isometric view of a small tray with spacers.

FIG. 22 is a diagrammatic isometric view of a small tray with plates and screws of different sizes and attachment screws.

FIG. 23 is a diagrammatic isometric view of a small tray with integral holder/connectors of different sizes.

FIG. 24 is a diagrammatic isometric view of a small tray with implants of different sizes.

FIG. 25 is a flow diagram of a method for assembling a spinal implant system.

### DESCRIPTION OF THE EMBODIMENTS

The following detailed description is provided to enable those skilled in the art to make and use the described embodiments set forth. Various modifications, equivalents, variations, and alternatives, however, will remain readily apparent to those skilled in the art. Any and all such modifications, variations, equivalents, and alternatives are intended to fall within the spirit and scope of the present invention defined by the below listed patent claims.

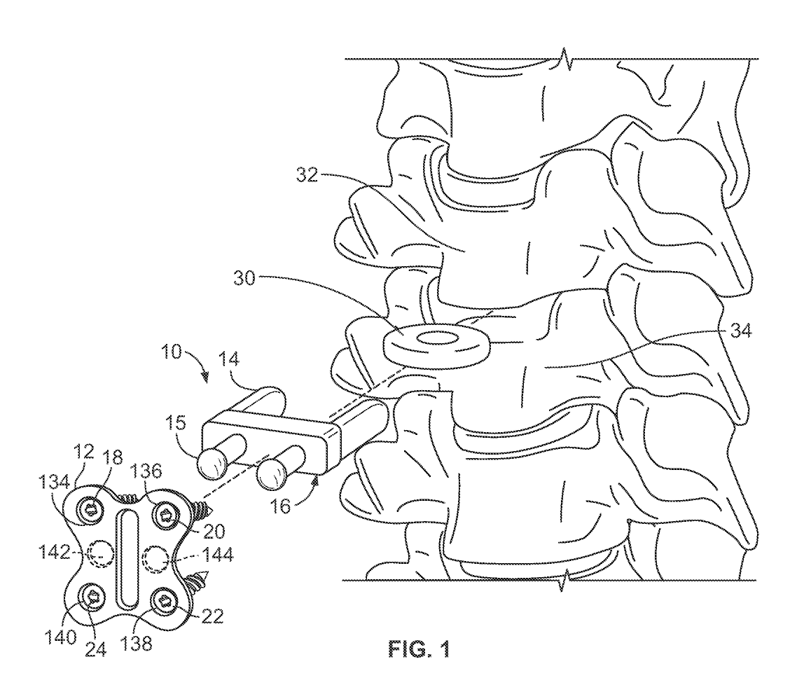

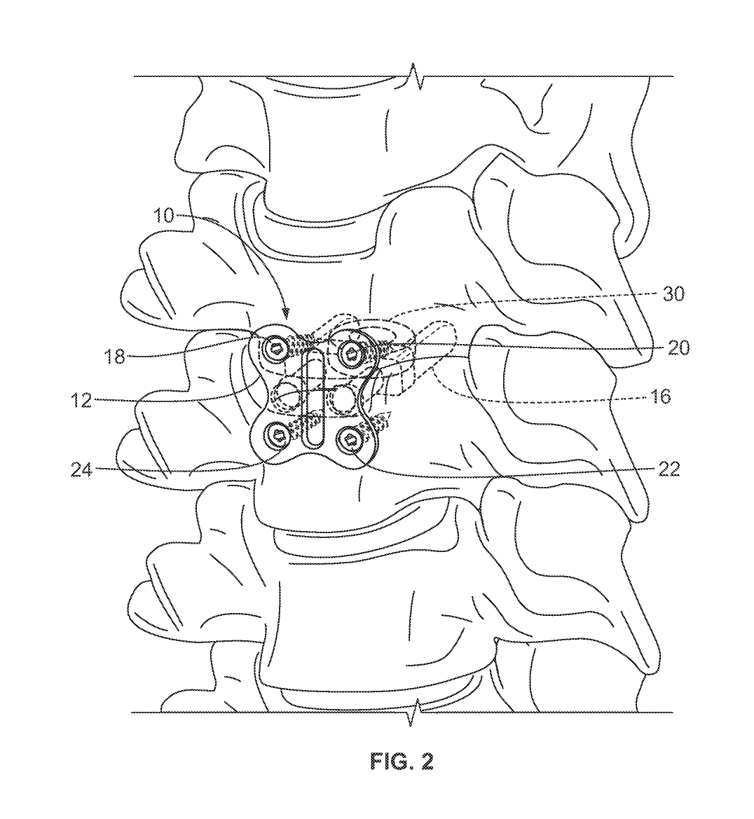

Referring now to FIGS. 1 and 2, there is shown a preferred embodiment of a spinal implant system 10, shown in an exploded view in FIG. 1, and a connected view in FIG. 2. The spinal implant system 10 includes a vertebrae plate 12, an implant holder 14 and a connector structure 15, which are in a preferred version an integral holder/connector 16, and four vertebrae screws 18, 20, 22, 24. A disk shaped implant 30 is illustrated being inserted between two vertebrae 32, 34 in FIG. 1, and completely inserted in FIG. 2. In the alternative, the holder and connector structure may be separate elements that are connected instead of being integral.

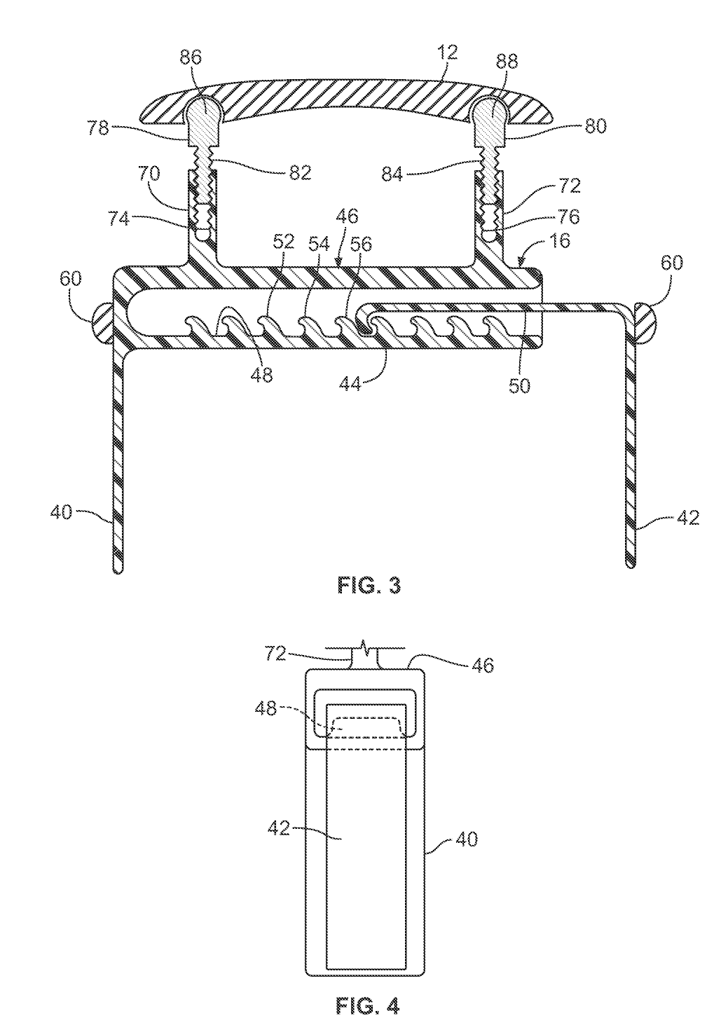

The integral holder/connector 16 includes flexible holder leg portions 40, 42, FIGS. 3 and 4, for grasping or holding the implant 30 during a surgical procedure involving vertebrae 32, 34. The implant may include whatever material a surgeon desires to use, such as auto graft (a patient's own tissue), allograft (tissue taken from another), or synthetic or metal cages. The holder 14 may include a ratchet mechanism 44 integral with the legs 40, 42 and forming a base member 46 where the rack portion 48 of the ratchet is integral with the left leg 40 and the pawl portion 50 is integral with the right leg 42 (as viewed in FIG. 3). The rack includes a plurality of teeth, such as the teeth 52, 54, 56. A surgeon may use a forceps instrument 60 to squeeze the rack and pawl sufficiently that the legs 40, 42 hold the implant 30 when the system is moved from a workstation, described herein below, and inserted into the patient's spine. By squeezing the ratchet mechanism 44 with the forceps, the pawl 50 moves from rack tooth to rack tooth until a proper hold is made between the legs 40, 42 and the implant 30. It is understood that once the pawl is moved past a rack tooth, the pawl cannot move in an opposite direction and the space between the two arm portions cannot be made larger because the ratchet is movable in one direction only. The connector section of the integral holder/connector 16 illustrated in FIG. 3 includes two upstanding arms 70, 72, each arm having an internal screw thread 74, 76. Extension arms 78, 80, each having a threaded base 82, 84 and a rounded head 86, 88, are adjustably connected so that the distance from the base member 46 and the implant to the plate 12 may be adjusted. As shown, a lower portion of the connector section is integral or connected to the holder and the opposite or upper portion of the connector is engageable with the plate 12.

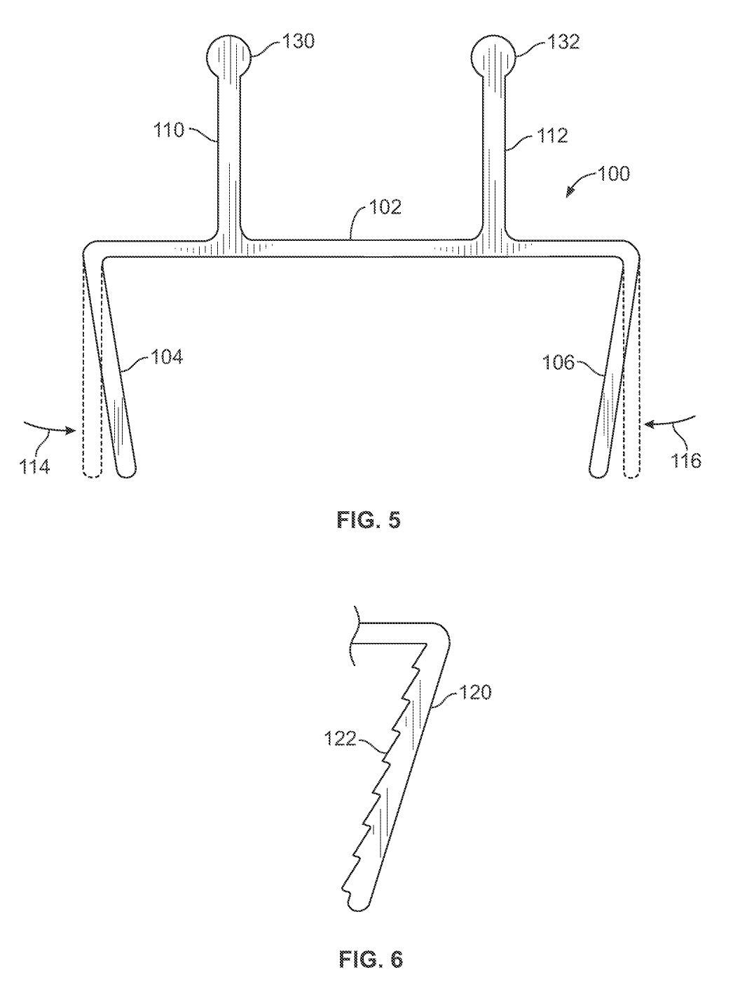

Alternative embodiments of the integral holder/connector are illustrated in FIGS. 5 and 6. For example, an integral holder/connector 100 is illustrated in FIG. 5, where there is a base member 102 and two downwardly extending (as seen in the figure) flexible holder legs 104, 106. As with the integral holder/connector 16, the integral holder/connector 100 includes two upwardly extending connector arms 110, 112. The holder legs may be set to converge in a relaxed position, shown in solid lines, and are resilient so as to spread apart, shown in dotted lines, when the legs engage an implant. The result is an inward biasing or squeezing force symbolized by arrows 114, 116 for gripping the implant. Illustrated in FIG. 6, is a variation leg portion 120 very similar to the version shown in FIG. 5, but an inside facing surface 122 is serrated for increasing a grip on an implant. Only one leg 120 is shown (the other leg is understood to be a mirror image) and it is set in a converging attitude when relaxed. In the alternative embodiments, illustrated in FIGS. 5 and 6, the connector arms 110, 112 are fixed so that the distance from rounded heads 130, 132 to the base member 102 is fixed. However, an instrument case (see FIG. 23) used by a surgeon may have a series of integral holder/connectors where the lengths of the connector arms 110, 112 vary in increments so that the surgeon measures the distance needed and picks the integral holder/connector with the correctly size connector arms.

The vertebrae plate 12 FIGS. 1 and 2 is formed of a suitable material, usually titanium, and includes four openings 134, 136, 138, 140 for the four screws. A plate may be made with two openings for two screws, if desired. Two recesses 142, 144 are provided to mate with the round heads 86, 88 of the connector arms, either with a “snap fit” or as a detent.

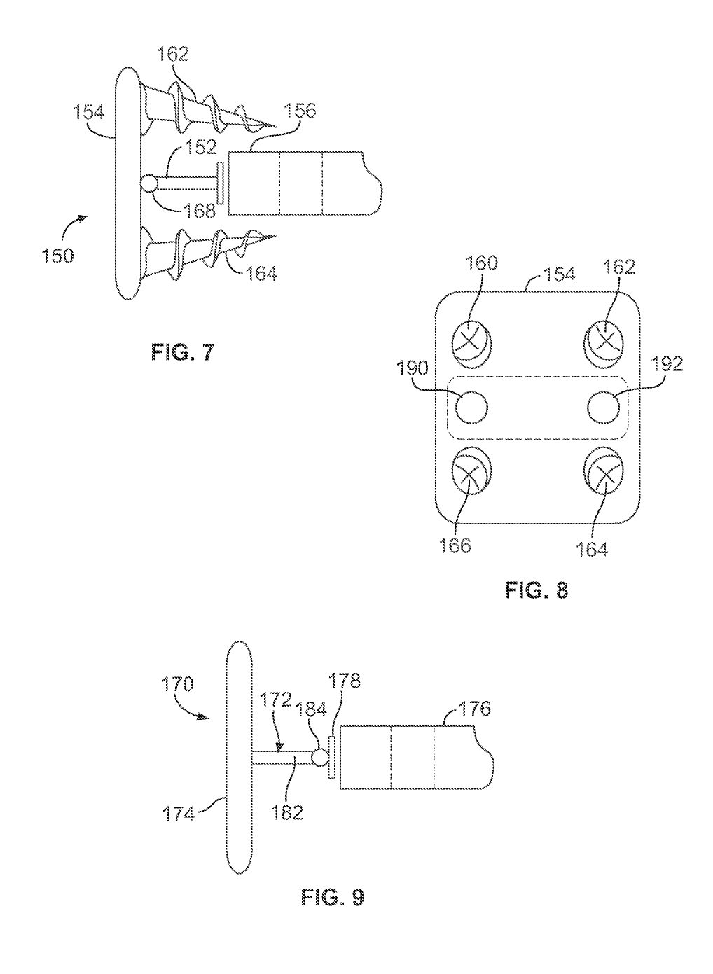

Referring now to FIGS. 7 and 8, there is shown diagrammatically a modular spinal fusion system 150 including a graft holder/connector 152 that may be connected to a stabilization or spinal plate 154 and to a spinal implant 156. The plate is connected to four vertebrae screws 160, 162, 164, 166. At the junction of the plate 154 and the holder/connector 152 is a pivot 168. The surgeon, for example, may, as mentioned, choose the implant 156, and all of the implants shown in other figures, from an auto graft, an allograft, or a synthetic or metallic cage. The plate 154 may be static or dynamic and the vertebrae screws may be fixed or multi-axial. Referring now to FIG. 9, a slightly different modular spinal fusion system 170 is shown diagrammatically (but without vertebrae screws) with a holder/connector 172 connected to a plate 174 and an implant 176. At a junction of a base 178 of the holder/connector and an arm 182 of the holder/connector 172 is a pivot 184. The plate 154, FIG. 8, may have two recesses 190, 192 to which an integral holder/connector may be snapped as mentioned above in relation to FIG. 3.

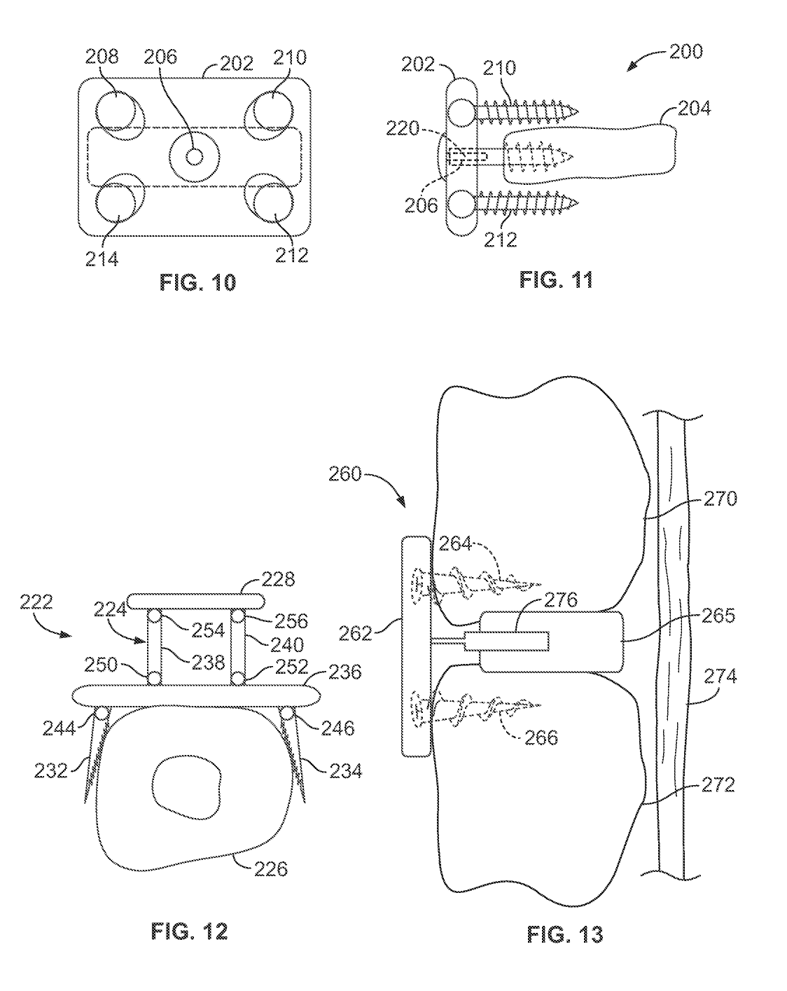

Another modular spinal implant system 200 is diagrammatically illustrated in FIGS. 10 and 11, and includes a plate 202, an implant 204, an adjustable holder/connector 206 and four adjustable vertebrae screws 208, 210, 212, 214. The holder/connector 206 may have a telescopic stem 220. Yet another modular spinal implant system 222 FIG. 12, is shown diagrammatically, and includes a holder/connector 224 attached to an implant 226 and to a spinal plate 228. The holder/connector includes two implant attachment serrated legs 232, 234 pivotally connected to a holder base 236, and two pivotally connected arms or stalks 238, 240 that may be connected to the plate 228. The stalks 238, 240 may come in a variety of lengths to meet variations in patient anatomies, or the stalks may be telescopic.

Articulation elements, such as hinges 244, 246 may be used between the base 236 and the attachment legs 232, 234, and ball sockets 250, 252 may be used between the base 236 and the stalks 238, 240, and another set of ball sockets 254, 256 may be used between the stalks 238, 240 and the plate 228. The hinges and ball sockets may be interchanged. Regardless, the articulation elements allow adjustability at the junctions of the legs and the base, and at the junction of the base and the stalks, and the junction of the stalks and the plate. A single stalk may be used instead of two. Referring now to FIG. 13, still another spinal implant system 260 is illustrated and includes a stabilization plate 262, two vertebrae screws 264, 266, an implant 265 inserted between two vertebrae 270, 272 adjacent a spinal core 274, and a fixed holder 276. The plate 262 may be elongated to connect to multiple holders, and the holders may be adjustable and placed between vertebrae at multiple levels. In the modular spinal implant systems disclosed, the size of the holder and the connected implant are selected to match the patient's anatomy and the depth of implant.

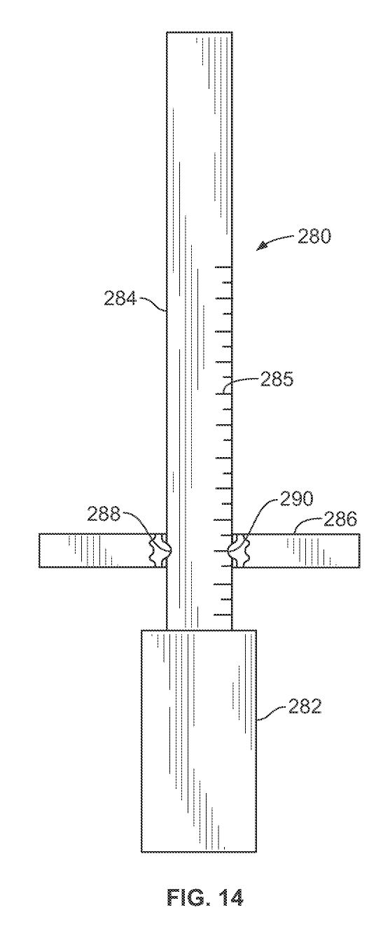

Referring now to FIG. 14, there is shown an instrument 280 for measuring the depth of an implant between vertebrae, and a trial spacer 282 attached to an end of the measuring instrument which is used to measure the gap between vertebrae and thereby the size of the implant to be used. The measuring instrument 280 includes a main vertical member 284 having measurement indicia 285 marked off along the member such that the depth of implant insertion may be determined. A cross member 286 is able to slide along the member 284 so as to quantify the depth of insertion. The cross member 286 is mounted to two small spring elements 288, 290 to allow smooth and easy movement. The trial spacer 282 may be one of a plurality of trial spacers each incrementally different in thickness from the others so as to give the surgeon the opportunity to accurately measure the space between the vertebrae at issue by using the spacers.

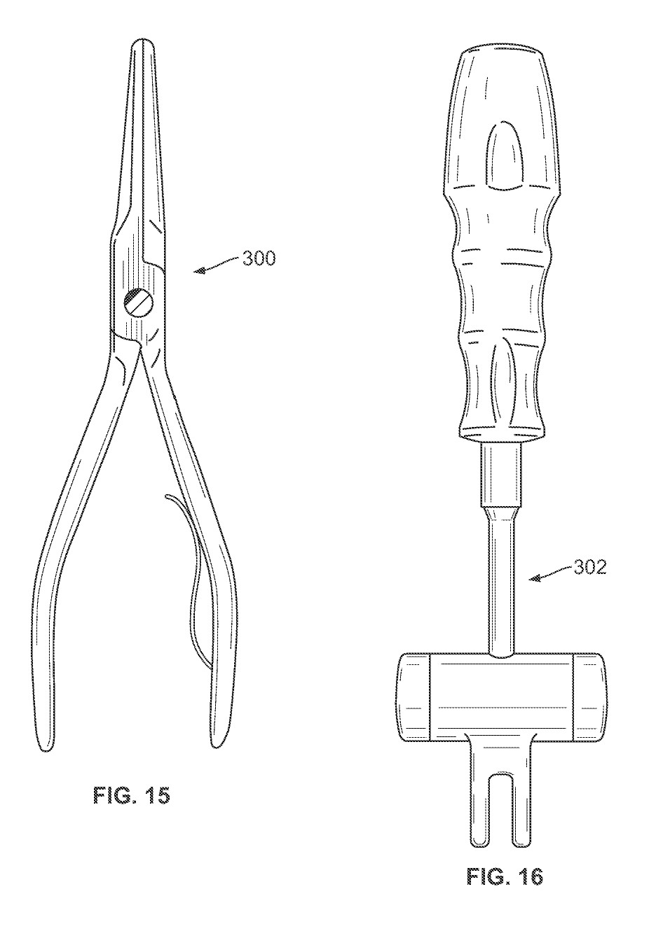

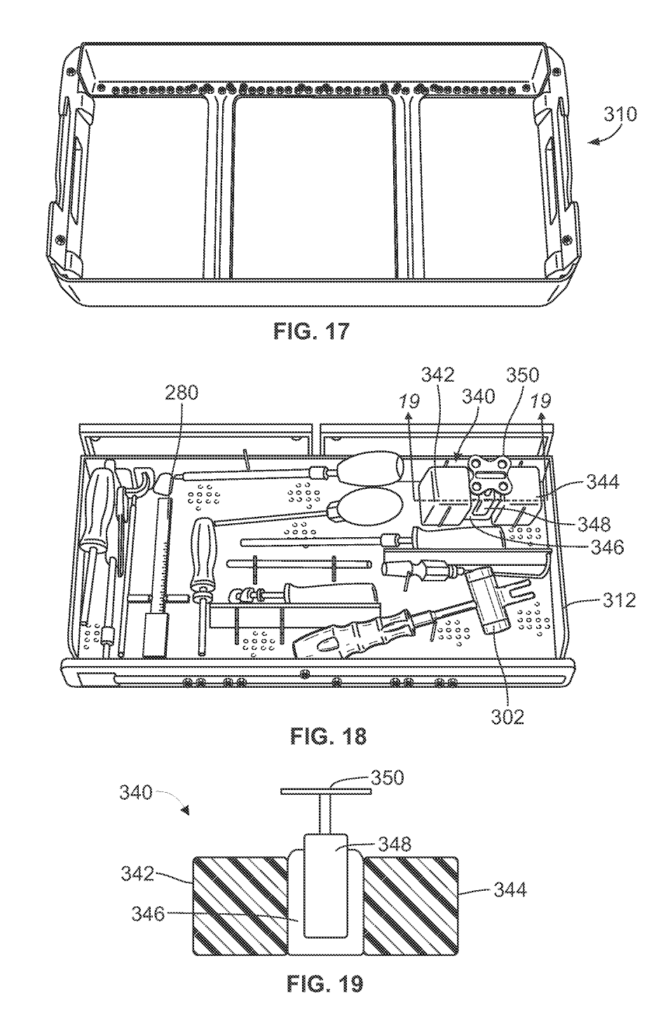

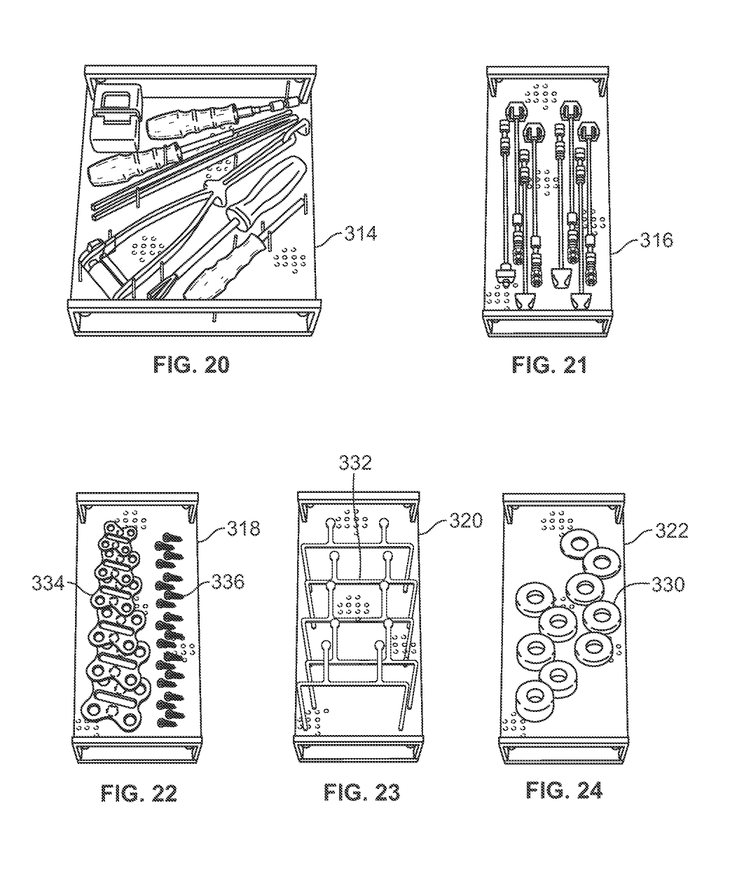

Examples of forceps 300 and a mallet 302 are illustrated in FIGS. 15 and 16, respectively. Referring to FIGS. 17-24, there is illustrated an instrument case 310 with removable trays 312, 314, 316, 318, 320, 322. By way of example, the large tray 312 and the smaller tray 314 may carry various tools used by the surgeon during a procedure. Other smaller trays 318, 320, 322 may carry items that are implanted or attached to a patient. For example, the tray 322, FIG. 24, may include a plurality of implants, such as the implant 330, each implant being of a different thickness. The tray 320, FIG. 23, may include a plurality of holder/connectors, such as the holder/connector 332, again in graduated sizes so that the surgeon may easily select the appropriate holder/connector to be used. The tray 318, FIG. 22, may include a plurality of plates and screws, such as the plate 334 and the screw 336, for again giving the surgeon the ability to select the appropriate size for the procedure being performed.

Special attention is directed to FIGS. 18 and 19, which illustrates an assembly or workstation 340 for the surgeon to use when bringing the modular implant system together. The workstation 340 may include two spaced apart soft resilient supports 342, 344 for holding in place an implant 346 that the surgeon has chosen after measuring the implant size required. After additional measurements the surgeon chooses a holder/connector 348, which is coupled to the implant. Finally, after additional determinations, the surgeon picks a desired plate 350 and screws and couples the plate to the holder/connector 348. After assembly of the three items, the implant 346, the holder/connector 348 and the plate 350, the assembled items are removed from the workstation 340 and placed into the patient.

The above described cervical implant or fixation assembly includes a combination interbody fusion device (the implant or graft and the holder/connector) and an anterior spinal plate designed for anterior application to the spine. The cervical fixation assembly allows lateral application to the spine to be achieved. The cervical fixation assembly is a modular unit and is easily mated to any one of a multiplicity of implant types, as mentioned. The cervical fixation assembly may have a plate component defined as static or dynamic, at either corner or anterior cortex of a vertebral body. The cervical fixation assembly includes the plate being pivotal and the implant holder/connector articulated to allow the plate to become situated in an anatomically correct position without pre-drilling. The cervical fixation assembly allows the plate component to mate with a variety of screw-plate locking mechanisms while the plate has a single or dual receptacle allowing attachment of the holder/connector. The cervical fixation assembly allows articulation between the plate and the implant to be static and defined by either a right angle or cephalo-cauded (head to toe) angulation. The cervical fixation assembly allows the plate to be rotated and mechanically fixed by stabilizing the holder/connector arms while the arms may have telescopic characteristics that allows for variable implant depth.

A method for the implantation of a spinal fixation assembly disclosed herein facilitates the sizing, orientation and implantation of the spinal implant assembly that is minimally invasive of the anatomical characteristics encountered, such as the vertebrae. The surgeon may attach the holder/connector to the implant while the implant may be at a workstation and stabilized through compression of the ratchet mechanism. The surgeon may also choose from a variety of implant materials. The holder/connector may be constructed from any one of a variety of materials, including metal or a suitable synthetic resin with varying degrees of flexibility. A routine discectomy or corpectomy may be performed using intervertebral pre-fabricated trial spacers, allowing for the determination of a total depth, wherein the spacers are used as trials so that ideal implant positioning is determined, the total depth from the edge of the vertebral body is measured, and a holder/connector and an implant are selected to match measurements. The plate length is selected based on experience and the surgeon's preference wherein the implant is attached to the holder/connector and the holder/connector to the plate such that the implant system becomes completely assembled and then set into position where the screws are placed into the vertebral bodies while a locking mechanism stabilizes the screws to the plate.

The spinal implant system described in detail above features a robust, but simply constructed system that is easy to assemble and insert.

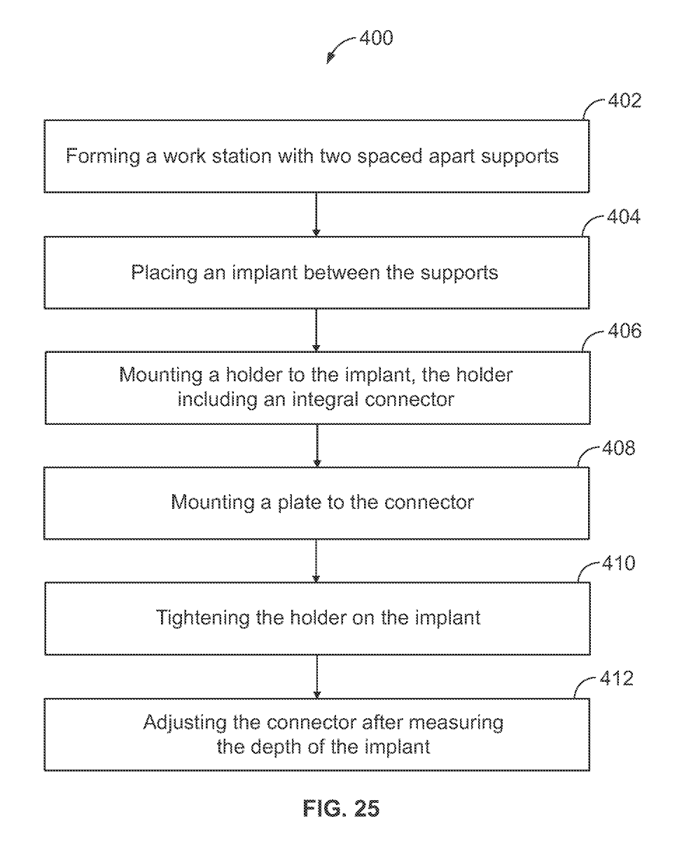

The present invention also includes a method 400, FIG. 25, of assembling a spinal implant system including the steps of forming a workstation with two spaced apart supports 402, placing an implant between the supports 404, mounting a holder to the implant, the holder including an integral connector 406, and mounting a plate to the connector 408. The method may also include the holder having a ratchet and including the step of tightening the holder on the implant 410, and the connector may have two adjustable arms and include the step of adjusting the connector arms after measuring the depth of the implant 412.

From the foregoing, it can be seen that there has been provided detailed features of the spinal implant system and a disclosure for the method of assembling the system. While particular embodiments of spinal implant systems have been shown and described in detail, it will be obvious to those skilled in the art that changes and modifications may be made without departing from the present invention in its broader aspects. Therefore, the aim is to cover all such changes and modifications as fall within the true spirit and scope of the claimed invention. The matters set forth in the foregoing description and accompanying drawings are offered by way of illustrations only and not as limitations. The actual scope of the invention is to be defined by the subsequent patent claims when viewed in their proper perspective based on the prior art.

What is claimed is:

1. A spinal implant system comprising:
a holder having two movable legs for engaging an implant;
a plate for engagement with vertebrae;
a connector connected at one end to the holder and extending a predetermined distance to connect at an opposite end to the plate; and
a plurality of screws positioned through the plate for fastening the plate to vertebrae wherein the implant, the holder, the connector and the plate are assembled before insertion into a patient.

2. The spinal implant system of claim 1, wherein:
the plate includes receiving structure to enable a snap fit with the connector.

3. The spinal implant system of claim 1, wherein:
the connector includes two adjustable arms.

4. The spinal implant system of claim 1, wherein:
the holder includes a ratchet.

5. The spinal implant system of claim 1, wherein:
the holder and connector are integral.

6. The spinal implant system of claim 1, wherein:
the holder legs are flexible.

7. The spinal implant system of claim 1, wherein:
the holder legs include serrations.

8. The spinal implant system of claim 1, wherein:
the holder and connector are integral;
the connector includes two adjustable arms; and
the holder includes a ratchet.

9. The spinal implant system of claim 1, including:
a workstation, the workstation having two spaced apart supports for holding an assembly of the implant, the holder, the connector and the plate.

10. The spinal implant system of claim 1, including:
an instrument for measuring depth of a spinal implant, the instrument having a stem marked with distance indicia, the stem being connectible to trial spacers and the instrument including a cross member movably connected to the stem.

11. The spinal implant system of claim 10, including:
a workstation, the workstation having two spaced apart supports for holding an assembly of the implant, the holder, the connector and the plate.

12. The spinal implant system of claim 11, wherein:
the holder and connector are integral;
the connector includes two adjustable arms; and
the holder includes a ratchet.

13. The spinal implant system of claim 11, wherein:
the holder and connector are integral; and
the legs of the holder are flexible.

14. The spinal implant system of claim 13, wherein:
the connector arms are fixed.

15. A spinal surgery set comprising:
a holder having a base and two extending legs, the legs being movable to enable the two legs to engage an implant;
a plurality of plates, each plate for engaging vertebrae;
a connector attached to the holder and extending a predetermined distance to engage one of the plurality of the plates;
a plurality of screws for fastening one of the plurality of plates to vertebrae;
a workstation, the workstation having two spaced apart supports of resilient material to enable the implant, the holder, the connector and a plate to be assembled; and
an instrument for measuring depth of a spinal implant, the instrument having a stem marked with distance indicia, the stem being connectible to trial spacers, and the instrument including a cross member movably connected to the stem.

16. The surgery set of claim 15, wherein:
the holder and connector are integral;
the connector includes two adjustable arms; and
the holder includes a ratchet.

17. The surgery set of claim 15, wherein:
the holder and connector are integral; and including
a plurality of integral holders and connectors, the connectors having arms wherein the length of the arms of the plurality of integral holders and connectors vary incrementally.

18. A method of assembling a spinal implant system comprising the steps of:
forming a workstation with two spaced apart supports;
placing an implant between the supports;
mounting a holder to the implant, the holder including an integral connector; and
mounting a plate to the connector.

19. The method of claim 18, wherein:
the holder includes a ratchet; and including the step of:
tightening the holder onto the implant.

20. The method of claim 19, wherein:
the connector includes two adjustable arms; and including the step of:
adjusting the connector arms after measuring the depth of the implant.
